(19) United States
(12) Patent Application Publication
(10) Pub. No.: US 2026/0363603 A1
(43) Pub. Date: Nov. 19, 2026

(54) SYSTEMS AND METHODS FOR ASSESSING VISUO-COGNITIVE FUNCTION

(71) Applicant: Okulo Ltd., Bristol (GB)

(72) Inventors: Stephanie Campbell, London (GB); Thomas Wemyss, Newbury (GB)

(21) Appl. No.: 19/121,877

(22) PCT Filed: Oct. 26, 2023
(86) PCT No.: PCT/EP2023/079959

§ 371 Date: Apr. 17, 2025

(30) Foreign Application Priority Data
Oct. 27, 2022 (GB) 2215905.7

(51) Int. Cl. A61B 5/00; A61B 5/16

(52) U.S. Cl. CPC A61B 5/4088; A61B 5/162; A61B 5/163; A61B 5/742

(57) ABSTRACT

A computer-implemented method for assessing at least one aspect of a user's visuo-cognitive function, the method comprising presenting a plurality of stimuli on a display screen of a handheld computing device, detecting a response, or a lack of response, from the user to the plurality of stimuli, determining at least one attribute associated with the user's responses, wherein each attribute is indicative of a level of difficulty associated with seeing or processing the plurality of stimuli, and recording the at least one attribute to enable an assessment of at least one aspect of a user's visuo-cognitive function.

### BACKGROUND

The present specification relates to computer-implemented methods and systems for assessing at least one aspect of a user's visual function.

The assessment of visuo-cognitive function is vital to understanding the health status and disease progression of a patient's eyes, visual and cognitive systems, and to understanding if they require visual correction, medical or lifestyle intervention. Visuo-cognitive function is defined as the ability to pick up and interpret/process visual information. As such, visuo-cognitive function may therefore be affected by deterioration of the associated visual system, or of deterioration of the cognitive function involved in that particular activity.

An accurate assessment of visuo-cognitive function is important in order to identify any eye or brain disease at the earliest opportunity, so that treatment can be started promptly. This is particularly difficult in the case of children and elderly patients, and also those with vision and/or cognitive impairments, where results of vision testing are inherently more variable. If a loss of vision goes undetected, it may in some cases quickly result in blindness. This can bring with it huge personal, social and economic consequences.

The measurement of visuo-cognitive or visual function is fraught with difficulty due to limitations of traditional testing charts, that lead to inherent variability and inaccuracies in measurements. Variability in testing occurs due to variations in lighting on printed charts, difference in test administration (for example, the amount of encouragement given to patients). Large discrete steps in the difficulty levels presented on eye charts leads to a loss of precision in measurement which may further enlarge the variability seen in results, masking the identification of small and early changes in visual status.

Common clinical visual function tests (such as the Snellen eye chart) rely upon recognition of letters. It can be difficult to get children, or those with learning disabilities or cognitive impairment to identify and verbalise a whole line of letters or symbols easily, which therefore create inaccuracies in results.

Initially, when big stimuli such as letters or symbols are presented, they are easily seen and identified by most users, and motivation is high. When progressing to the smaller letters or symbols during a traditional vision test, they become less easy for the user to see, and the user becomes less confident. If the user's attention drops off, a poorer level is recorded, and the clinician does not know if this is due to poor attention, or due to an underlying vision problem. When a patient is reading a regular eye chart, the result is recorded as how many letters/symbols they read ‘correctly', thus each letter/symbol identification has a binary outcome-either it is correct or incorrect. However, clinicians often report that those with early disease or blur are often able to identify symbols on the eye chart, but do so more slowly or report a greater perceived difficulty. Unfortunately, using traditional eye charts and scoring mechanisms, this subtle and nuanced 'difficulty’ information is lost, and consequently small changes in vision are not captured.

The present disclosure provides a computer-implemented method and system which seeks to remedy some or all of these deficiencies, increasing the potential accuracy and granularity in visuo-cognitive testing.

### SUMMARY

Aspects of the present disclosure are set out in the accompanying independent and dependent claims. Combinations of features from the dependent claims may be combined with features of the independent claims as appropriate and not merely as explicitly set out in the claims.

Understanding visual function and visual perception is a cornerstone of two medical specialities: ophthalmology and neurology. The present disclosure provides a method and system that is not limited to assessing standard visual acuity (synonymous with the letter chart), but can assess wider visuo-cognitive function of the user in a manner that can provide not just optical information, but information on neural processing of information from within the brain itself, i.e. ‘visuo-cognitive’. The presented disclosure describes a method and system that can preferably be carried out by a user at home and provides an indication of visuo-cognitive function that would otherwise only be available from equipment in specialist hospital clinics and vision labs.

According to a first aspect of the present disclosure, there is provided a computer-implemented method for assessing at least one aspect of a user's visuo-cognitive function, the method comprising presenting a plurality of stimuli on a display screen of a computing device or system. For ease of reference we will simply refer to this hereafter as a ‘device’. In a preferred embodiment, the computing device may comprise a handheld computing device.

The method may further comprise detecting at least one response, or a lack of response, from the user to the plurality of stimuli.

The method may further comprise determining at least one attribute associated with the user's at least one response, or the user's lack of response. Each attribute may be indicative of a level of difficulty associated with seeing or processing the plurality of stimuli. ‘Determining’ may comprise one or more of measuring, detecting or otherwise processing data relating to the at least one attribute. The term ‘attribute’ may be used interchangeably with ‘metric’.

The method may further comprise recording the at least one attribute to enable an assessment of at least one aspect of a user's visuo-cognitive function. The term ‘assessment’ may comprise measuring, recording and/or deriving data relating to the at least one aspect of the user's visuo-cognitive function. Thus, embodiments of the disclosure may comprise technical solutions for generating, measuring, processing and/or storing data relating to the at least one aspect of the user's visuo-cognitive function.

It will be appreciated that the term stimuli or stimulus may refer to any image, animation or visual object that can be presented on a display screen of a handheld computing device. In some embodiments a stimulus may comprise a plurality of images, animations or visual objects. Optionally, the term optotype or image may be used interchangeably with the term stimulus.

Optionally, the method may include detecting a response, or a lack of response, from the user to each stimulus presented on the display screen.

Optionally, a plurality of stimuli may be presented on the display screen at a given display time and the method may include detecting a response, or a lack of response, from the user to the plurality of stimuli. The plurality of stimuli may be repeatedly presented on the display screen and a response, or lack of response, from the user may be detected at each presentation.

It will be appreciated that the attribute may be determined for each response received. The attribute may be indicative of a level of difficulty associated with seeing or processing each stimulus.

In some embodiments, the attribute may be associated with the user's lack of response. For example, if the user does not respond to a stimulus (as discussed below) but does move closer to the screen, this may be detected as an attribute.

In one or more embodiments, the level of difficulty associated with one or more of the plurality of stimuli may be adjusted or altered when the plurality of stimuli is subsequently presented to the user. In some embodiments, the difficulty level associated with all of the stimuli in the plurality may be adjusted. The level of difficulty may be adjusted or altered based upon the attribute that was previously determined for the one or more stimuli. For example, if the previous presentation of the stimuli resulted in the user bringing the device closer to their eyes, and thus a distance attribute being detected or measured, larger versions of the one or more stimuli may be provided in the subsequent presentation. In another example, if the presentation of the stimuli results in the determination of a slow response time, in the subsequent presentation the stimuli may move or change colour or flash. The subsequently determined data relating to the attribute(s) can then be re-recorded. Thus, the method may comprise the step of (dynamically) changing the level of difficulty based on the at least one attribute that has been previously determined based on the user's response or lack thereof. One or more embodiments of the disclosure may be arranged to operate by starting with a high level of difficulty and making the one, some or all stimuli easier to see in at least one subsequent presentation to the user.

The plurality of stimuli may comprise a plurality of distinct or individual stimuli. In other embodiment, a block or section of text may be considered to be a single stimulus in the present disclosure. However, the present disclosure is not directed to measuring reading speed, thus reading speed is not an example of an attribute according to the present disclosure.

In the present disclosure, the assessment of at least one aspect of the user's visuo-cognitive function is not based only on whether or not the user can see each stimulus, as is common in the prior art, but on how the user is responding to the stimuli. Advantageously, in the present disclosure at least one attribute of the user's response is also determined, which therefore provides additional, and likely more subtle or nuanced, information upon which the assessment of visuo-cognitive function can be at least partially based.

The at least one attribute associated with the user's response to each stimulus may provide quantitative information related to the user's response (where previously this could only be recorded qualitatively). The at least one attribute may be any property associated with the user's response that may provide quantitative information indicative of visuo-cognitive function.

In some cases, monitoring the at least one attribute associated with the user's response to each stimulus can provide early warning or early indication of possible issues or declines in visuo-cognitive function, which may otherwise have been undetected.

Optionally, the at least one attribute may include one or more of response time or average response time, number of incorrect responses, pattern or order of the responses received, amount of movement of the handheld computing device relative to a user's eye when the response is received, distance of the handheld computing device relative to a user's eye when the response is received. It will be appreciated that this is not an exhaustive list. The attributes are discussed in more detail below.

The method may comprise providing an assessment of at least one aspect of the user's visuo-cognitive function at least partially based on the at least one attribute.

The method may comprise providing an assessment of at least one aspect of the user's visuo-cognitive function based on the at least one attribute and the response, or lack of response, to each stimulus. For example, the assessment may be based on whether or not the response indicates that the user can see each stimulus, in addition to the at least one attribute recorded for the user's responses.

Optionally, detecting a response from the user comprises receiving an input from the user indicating a proposed location of the respective stimulus on the display screen. In some embodiments, the user's input may be received on a user input device.

The at least one attribute may comprise an accuracy of the proposed location compared to an actual location of the stimulus on the display screen.

The method may comprise determining whether the response from the user indicates that the user has seen the respective stimulus. This may be done by determining whether the proposed location of the stimulus is within a predetermined range of an actual location of the stimulus on the display screen.

Optionally, the at least one attribute comprises response time or average response time. Determining the at least one attribute may comprise determining a response time for each response, wherein the response time is the time elapsed between a display time when the stimulus is presented on the display screen and a time that the response from the user is detected.

It will be appreciated that the term reaction may be used interchangeably with response throughout the present disclosure.

It will be appreciated that the time that a user takes to respond to a stimulus can be an indicator of visuo-cognitive function. A shorter response time can indicate that a stimulus is easier for the user to see. Thus, the response time itself can provide useful information regarding visuo-cognitive function.

In the prior art, a user's response time is often measured only for the purpose of assessing whether the user has ‘seen’ the stimulus or not, as if the response time is above a predetermined limit the user is determined not to have seen the stimulus. In the prior art, the response time itself is not recorded or assessed as providing useful information regarding visuo-cognitive function.

For example, measuring and recording the response time of the user to each stimulus may provide an early indication of a decline in visual function. This is because the user's response times may increase, indicating that the stimuli are harder to see, even though the user may still be correctly identifying or reacting to the stimuli. As such, this may be missed by a system that simply detects whether or not the user can see the stimulus, without recording the response time as a qualitative measure of how well (or quickly) the user sees each stimulus.

Optionally, the method may comprise determining an average response time of the user. For example, a first overall average response time may be determined for the user's right eye and a second overall average response time may be determined for the user's left eye. In some embodiments, an overall average response time may be determined for both eyes together. Optionally, the overall average response time for the right and/or left eye may be compared to the overall average response time for both eyes.

Whilst the at least one attribute of the user's response may provide useful information on its own (i.e. as an instantaneous or one-off assessment of visual function), in some embodiments the at least one attribute may be monitored over time. Thus, it may be a trend in the at least attribute of the user's responses that may provide useful information regarding visual function.

The method may include adjusting the determined response time to account for a learning effect. It will be appreciated that when a user is more familiar with a test, or the mechanics of the test, their response times generally improve (decrease). This can provide false indication that the user is finding the stimuli easier to see. As such, it may be desirable to account for this learning effect.

Optionally, the method includes recording a value indicative of a number of assessments (i.e., the number of times that the visual test or program has been run or completed by the user) that have been completed by the user, or the calendar time period over which the user has undertaken assessments, or a number of stimuli that have been presented to the user. It will be appreciated that each session wherein stimuli are presented on the display screen may be defined as an assessment. The method may include applying a scaling formula to the determined response time to account for the learning effect, wherein the scaling formula is based at least in part on the value.

Optionally, the method may comprise recording the number of assessments that have been completed by the user, or the number of stimuli that have been presented to the user.

The method may comprise applying a scaling factor to the determined response time to account for a learning effect, wherein the scaling factor is based on the number of assessments that have been completed by the user or on the number of stimuli that have been presented to the user.

The method may include comparing the determined at least one attribute against stored data. Optionally, the method may include comparing the determined at least one attribute against stored data for said user, such as data stored for the user from previous assessments.

Thus, the method may comprise monitoring the at least one attribute associated with the user's responses over time. The method may include monitoring the determined at least one attribute to enable assessment as to whether there is a change in visuo-cognitive function of the user over time. A significant change in the at least one attribute compared to the user's average data (or previous data), even if the user is still performing relatively well in terms of seeing the stimuli, may be indicative of an issue or decline in visuo-cognitive function.

Optionally, the method may include comparing the determined at least one attribute against an age normative database comprising age-matched average data. Thus, the method may comprise monitoring the user's responses over time compared to an age-matched average ‘standard’.

Optionally, the method comprises storing a date and time of the assessment. The method may include comparing the determined at least one attribute to stored data from at least one previous assessment executed by the user.

Optionally, determining the at least one attribute comprises determining any incorrect responses received from the user. Each incorrect response may indicate that the user has not seen the stimulus, or has not seen one of the plurality of stimuli.

For example, the method may comprise detecting the number of mistakes made by the user. If the user displays hesitancy, is looking all around the screen prior to looking at the stimulus, or is pressing all around the screen at locations that do not correspond to one of the stimuli, then this may indicate that the user is finding it harder to see the stimuli compared to a user who makes fewer incorrect responses (or mistakes).

Optionally, the method may include determining the type of mistakes or incorrect responses made by the user. The method may comprise recording at least one of a time of receipt and a location on the display screen of each incorrect response received from the user.

The method may include determining an accuracy of the responses received from the user, based on the number of incorrect responses received. For example, a ratio of the number of incorrect responses to ‘correct’ responses received, wherein a correct response is defined as a response where it is determined that the user has seen the stimulus (e.g. looked at or pressed the location of the stimulus on the display screen).

Optionally, determining the at least one attribute comprises measuring movement of the handheld computing device when each response from the user is detected. For example, the movement may be detected by an accelerometer in the handheld computing device, or a camera.

Optionally, determining the at least one attribute comprises determining a distance between the user, or the user's eye, and the display screen when each response from the user is detected. The distance may be determined using a camera, wherein the camera may be coupled to, or integrated with, the handheld computing device. If the user has moved the display screen closer to their eye, or further away from their eye, in order to see a given stimulus, then this provides additional information indicative of visual function.

Optionally, determining the at least one attribute comprises determining an order or pattern in which the user's responses to the plurality of stimuli are received. For example, the at least one attribute may be an indication of the order in which the plurality stimuli are determined to be seen by the user, or the order in which the responses indicating that the user has seen each stimuli are received.

The order in which the user sees the stimuli presented on the display screen, or indicates that they have seen the stimuli, may provide useful information indicative of at least one aspect of visual function. For example, users with poor visual function often interact with stimuli in order of how difficult the stimuli are to see, starting with the easiest to see stimuli first. Thus, the order in which the user interacts with the stimuli can provide additional information that may indicate how easily the user can see each stimulus.

For example, in comparison to a user with poor vision, a user with good vision may start interacting with all stimuli on one side of the display screen and move progressively to the other side of the display screen, regardless of how easy or hard to see each stimulus is.

Optionally, determining the at least one attribute comprises determining a location on the display screen or device at which the user was targeting their gaze when each response from the user is detected.

Optionally, determining the at least one attribute comprises determining the user's facial expression during the time in which the user is identifying and/or responding to the stimulus.

The plurality of stimuli may comprise stimuli which are presented specifically for the purpose of assessment of visuo-cognitive function. Additionally or alternatively, the plurality of stimuli may comprise display elements typically encountered during the normal usage of the handheld computing device, or modifications thereof.

Each stimulus may have at least one respective visual property. The at least one visual property may have a predetermined value selected to assess at least one aspect of visuo-cognitive function.

The at least one visual property may comprise one or more of: position on the display screen; contrast; colour; level of detail; shape; size; or movement of the stimulus on the display screen.

Optionally, the at least one visual property of the stimulus has a predetermined value selected to assess at least one aspect of visual function. Optionally, the plurality of stimuli do not have the same predetermined value for the at least one visual property, thus the visual property may vary across the plurality of stimuli. The at least one visual property of the stimulus may change during the assessment.

Optionally, the at least one visual property of the stimulus comprises one or more of: position on the display screen (including but not limited to position relative to other stimuli on the display screen); contrast; colour; level of detail; shape; size; or movement of the stimulus on the display screen.

Optionally, the at least one aspect of visuo-cognitive function comprises one or more of: visual acuity; central visual field; contrast sensitivity; stereopsis; colour vision; detection acuity; resolution acuity including spatial resolution or identification of static of dynamic directionality; recognition acuity; vernier acuity; hyperacuity; temporal acuity; and/or spectral acuity.

Optionally, a plurality of aspects of visuo-cognitive function are measured using the plurality of stimuli. Thus, a plurality of aspects of visuo-cognitive function may be measured simultaneously.

Optionally, two or more of the plurality of stimuli may be presented on the display screen at the same time. Optionally, only one stimulus may be presented on the display screen at any given time. Thus, the plurality of stimuli may be presented on the display screen at the same time, or at different times.

Optionally, the plurality of stimuli are the same stimulus, wherein one or more of the visual properties of the stimulus are adjusted upon each presentation (or version) of the stimulus.

Optionally, the plurality of stimuli comprises at least two different stimuli, wherein one or more of the visual properties of each stimulus are adjusted upon each presentation.

Optionally, the method comprises further comprises repeatedly presenting one or more of the plurality of stimuli on the display screen. One or more of the visual properties of each stimulus may be adjusted upon each presentation of the stimulus.

Optionally, the stimuli are presented, or repeatedly presented, on the display screen until a threshold is determined for the at least one aspect of the user's visuo-cognitive function.

The method may comprise comparing the at least one attribute associated with the user's response to a stimulus near the user's threshold for a given aspect of visuo-cognitive function, to the at least one attribute associated with the user's response to a stimulus above the user's threshold for the same aspect of visuo-cognitive function.

It will be appreciated that stimuli that are above the user's threshold of visual function are capable of being seen by the user. In contrast, stimuli that are below the user's threshold of visual function are not capable of being seen by the user.

Optionally, the display screen comprises a touchscreen, wherein detecting a response from the user comprises detecting a point of contact from the user on the touchscreen.

Optionally, detecting a response from the user comprises monitoring movement of the user's eye using a camera. Thus, the handheld computing device may be configured to use preferential looking techniques to assess whether the user has seen each stimulus.

Preferably, each stimulus is presented on the display screen within a gamified environment. This may ensure that the assessment is more interesting and engaging, particularly for children, which can provide improved results.

Optionally, the method may include storing the determined at least one attribute associated with the user's response to each stimulus in a memory. Optionally, all data resulting from the assessment is stored in the memory.

Optionally, the method may include transmitting the determined at least one attribute associated with the user's responses to a remote server and/or a remote electronic device via a communication channel. The communication channel may be a wireless communication channel.

The method may comprise executing the assessment for a first eye of the user with the user's second eye being covered, and then repeating the assessment for the user's second eye with the first eye being covered.

Optionally, the method may comprise comparing the determined at least one attribute associated with the user's response to each stimulus for the first eye to the determined at least one attribute associated with the user's response to each stimulus for the second eye.

The method may comprise determining a plurality of attributes associated with the user's response to each stimulus.

Optionally, determining whether or not the response from the user indicates that the user has seen the stimulus may enable a primary assessment of visual function. Determining the at least one attribute associated with the user's response to each stimulus may enable a secondary assessment of visual function. The at least one aspect of visual function assessed by the primary assessment of visual function and the secondary assessment of visual function may be the same, or different.

In a second aspect, the present disclosure provides a handheld computing device for assessing at least one aspect of a user's visuo-cognitive function, the handheld computing device comprising a display screen, a processor and memory including executable instructions that, as a result of execution by the processor, causes the handheld computing device to perform the computer-implemented method of any embodiment or example of the first aspect of the disclosure.

The display screen may comprise a touchscreen. To detect a response from the user the handheld computing device may be configured to detect a point of contact from the user on the touchscreen.

Optionally, the handheld computing device comprises a camera. The camera may be configured to monitor movement of the user's eye to detect a response from the user. Thus, the handheld computing device may be configured to use preferential looking techniques.

Optionally, the handheld computing device may be a mobile phone, smart phone or a tablet computer.

### BRIEF DESCRIPTION OF THE DRAWINGS

Embodiments of this disclosure will be described hereinafter, by way of example only, with reference to the accompanying drawings in which like reference signs relate to like elements and in which:

FIG. 1A is an illustration of a display screen of a handheld computing device executing a vision assessment according to an embodiment of the present disclosure;

FIG. 1B is an illustration of a display screen of a handheld computing device executing a vision assessment according to another embodiment of the present disclosure;

FIG. 2 is a block diagram illustrating a handheld computing device according to an embodiment of the present disclosure;

FIG. 3 is a flowchart illustrating a computer-implemented method according to an embodiment of the present disclosure; and

FIG. 4 is a flowchart illustrating a portion of a computer-implemented method according to another embodiment of the present disclosure.

### DETAILED DESCRIPTION

Currently, vision is usually measured as a threshold determined from a difficulty level, for example in the case of the traditional visual acuity chart wherein the smallest row of letters/symbols that a patient can discern is taken as the lower threshold of visual ability. However, it is now widely accepted that visual acuity does not always identify visual deterioration at the earliest possible stage. It is likely that traditional thresholding of visual measurement is too coarse and misses out the subtleties of visual deterioration that the patient experiences.

Visual acuity represents the very central part of the retina and macula, called the fovea, which is fine tuned to seeing detail. Subtle deteriorations may occur near to the fovea and thus be ‘missed’ by traditional visual acuity measurements, which focus on the detail in the very centre of the vision.

Since the wider macula is used for visual stabilisation during image detection and recognition, a defect in part of the surrounding macula may preferentially affect the locating and the stabilisation of the image. Such a defect will be mitigated by the nearby healthy retina, and as such often returns a normal vision result. However, the defect will impact on the ease of seeing, likely prolonging the time to correct identification of the image. This is likely to manifest as a time delay and/or unconfident and/or incorrect responses from the patient.

Depending upon the location and/or the nature of disease in the visual pathway, visual deterioration may also vary amongst different aspects of visual function. For example, visual acuity is maintained in recovered optic neuritis, despite retinal function being impaired by more sensitive electrophysical measures. In early glaucoma and early cataract, normal visual acuity is often maintained despite a reduction in sensitivity to lower contrast stimuli, and also associated more variability in response. Such measures could be used in themselves to map progression of, or recovery from disease.

In fact, certain aspects of visual function may not only indicate retinal changes, but also neurological changes in associated pathways.

The present disclosure seeks to remedy the deficiencies of many prior art visual assessment methods and systems.

Embodiments of this disclosure are described in the following with reference to the accompanying drawings.

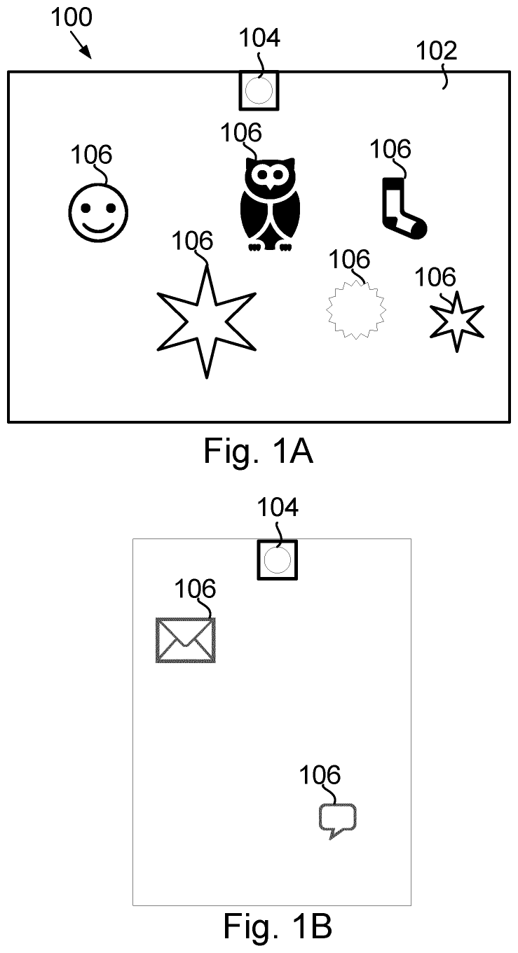

FIG. 1A shows a representation of a handheld computing device 100 comprising a display screen 102 and a camera 104. In some embodiments, the handheld computing device 100 may not comprise a camera 104, or the camera 104 may not be integral to the handheld computing device 100. In some embodiments, the camera 104 may be a webcam or other camera that may be coupled to the handheld computing device 100 via wired or wireless connection. The display screen 102, or at least a portion of the display screen 102, may be a touchscreen.

The handheld computing device 100 as shown in FIG. 1A is executing an assessment of a user's visuo-cognitive function. On the display screen 102 six stimuli 106 are presented. Each stimulus 106 may have at least one visual property selected to measure at least one aspect of visuo-cognitive function. The at least one visual property may include, but is not limited to, the position, contrast, colour, level of detail, shape, size, or movement of the stimulus on the display screen. The display screen 102 may be configured to present a background or background image, which may be changed during the assessment.

In FIG. 1A, for simplicity, the stimuli 106 are represented as different shapes. It will be appreciated that the stimuli are not limited to the shapes or forms shown in FIG. 1. Instead, any visual stimulus may be presented on the display screen. In addition, although six stimuli 106 are shown in FIG. 1, any number of stimuli may be presented on the display screen 102 either simultaneously, or in succession.

In some embodiments, the same stimulus 106 may be repeatedly presented to the user on the display screen 102, with at least one visual property of the stimulus changing upon each presentation. In some embodiments, one or more of the stimuli 106 may be configured to move on the display screen 102. In some embodiments, one or more of the stimuli 106 may be stationary on the display screen 102.

Preferably, the stimuli 106 are presented within a gamified environment. As such, the assessment may take the form of a computer game played by the user on the handheld computing device 100. This may make the assessment more interesting and engaging, particularly for children, which may improve the results of the assessments as the user's attention is maintained for longer than a traditional eye test.

The handheld computing device 100 may be, but is not limited to, a mobile phone or smart phone or a tablet computer. The handheld computing device 100 comprises memory and at least one processor.

The assessment or vision test represented in FIG. 1A may be executed by an application or computer program that is downloaded and installed on the handheld computing device 100. The computer program may be defined by a set of computer-executable instructions stored in the memory of the handheld computing device 100.

In FIG. 1B, an alternative embodiment is represented wherein the assessment is carried out using stimuli which include display elements typically encountered during the normal usage of the handheld computing device 100, or modifications thereof. In some embodiments, the assessment may be executed as a ‘background’ application during normal operation of the handheld computing device 100, such that the user may not be aware that an assessment is being carried out. Thus, once the application is installed on the handheld computing device 100, the user may not have to open the application to initiate an assessment. Instead, the assessment may initiate or run when the user is performing given tasks on the handheld computing device 100.

As shown in FIG. 1B, the stimuli 106 may include an email notification icon, and another symbol indicative of a notification or alert (e.g. message or call received). The method of the present disclosure may include detecting the user's response to these stimuli 106 that are encountered during use of the handheld computing device 100 and determining at least one attribute associated with the user's responses, wherein each attribute is indicative of a level of difficulty associated with seeing or processing the plurality of stimuli. The at least one attribute is then recorded to enable an assessment of at least one aspect of a user's visuo-cognitive function.

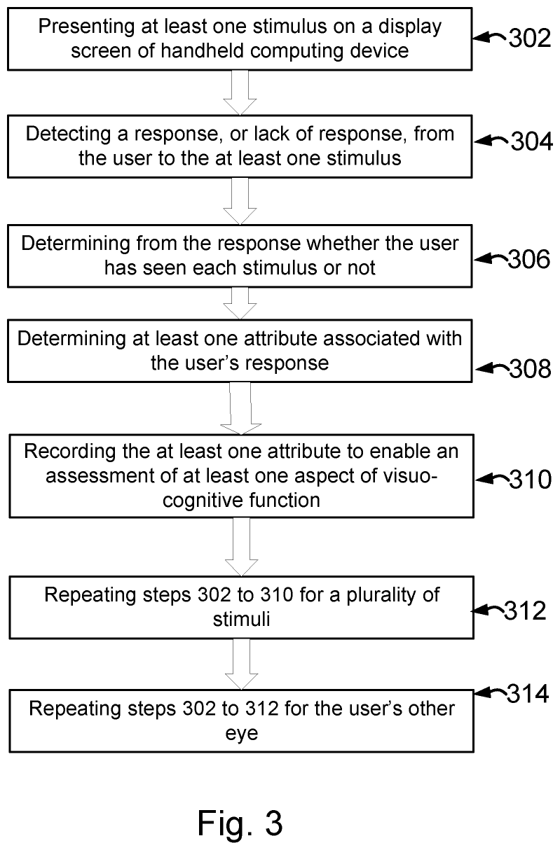

FIG. 3 is a flowchart illustrating a method of assessing at least one aspect of visuo-cognitive function according to an embodiment of the present disclosure. An initial step of the method, not shown in FIG. 3, is that the user may cover one of their eyes, for example using an eye-patch or a screen. The at least one aspect of visual function may therefore be separately assessed for each eye. The user may then initiate the assessment, for example, by entering their personal details (username, password, etc.) or biometric detail(s) such as a fingerprint, to log in to a user account. This may allow the assessment to be linked to the user and may ensure that the correct person is sitting the assessment. In addition, the assessment may be personalized to the particular user.

At step 302 the method comprises presenting at least one stimulus on a display screen of a handheld computing device 100, 200. In some embodiments, only a single stimulus may be presented on the screen at a given time, in other embodiments multiple stimuli may be presented at the same time. It will be appreciated that multiple icons or images could also be considered to be a single stimulus, if they are presented at the same time. Each stimulus may have at least one respective visual property. The visual properties of each stimulus may be defined by the computer program, or the algorithm controlling the assessment.

At step 304 the method comprises detecting a response, or lack of response, from the user to the at least one stimulus. The response may also be referred to as a reaction. The method may comprise detecting a plurality of responses to a plurality of stimuli, or a single response to a plurality of stimuli. It will be appreciated that, if the user is not capable of seeing or distinguishing the stimulus, then they may have no reaction or response to the stimulus. As such, step 304 may comprise detecting the absence of a response or reaction from the user.

In some embodiments, the method then determines whether the user response indicates that the user has seen the at least one stimulus, or not (step 306). For example, is the response a ‘correct’ response, such as touching the location of the stimulus or looking at the stimulus. This step may not be included in all embodiments, particularly in embodiments focused on the attributes of the user's responses.

There are a number of different ways in which the handheld computing device 100 may detect the user's response. In some embodiments, the handheld computing device 100 comprises a camera 104 configured to monitor the user's eye movement. The camera 104 may be able to determine whether or not the user has seen each stimulus presented on the display screen based on the user's eye movements. These techniques are known in the field of vision assessment. This method may be preferred for young children, or people with physical or cognitive impairments that may make verbal or physical responses difficult. As such, the assessment may not require the user to make a choice between different stimuli, or to make any kind of decision verbally or physically. Instead, the user may simply have to look at the stimulus if they can see it. Thus, the present disclosure may use eye tracking and/or gaze tracking to, for example, assess preferential looking.

In another embodiment, the handheld computing device 100, 200 may be configured to receive an input from the user (other than their eye movement). In some embodiments, the display screen 102 may be a touchscreen. The user may be required to press, tap, or touch the touchscreen at the location where they believe a stimulus to be currently presented. If the location at which the user presses, taps, or touches the touchscreen is within a predetermined tolerance of the correct location of a stimulus on the display screen, then it may be determined that the user has seen that stimulus. In other embodiments, the user input may be received on a user input device that is not the display screen 102, for example a keypad, controller, or other input device.

It will be appreciated that the method of detecting the user's response can vary between different users, depending on their needs and preferences. For example, the method of detecting the user's response may be associated with the user's account or profile.

At step 308, the method comprises determining at least one attribute associated with each response from the user. Each attribute is indicative of a level of difficulty associated with seeing or processing the stimuli. The attribute therefore provides additional, preferably qualitative, information as to how the user has seen or reacted to the stimulus, in addition to the yes or no decision as to whether or not the user has responded to stimulus. This can provide very useful information that can provide early warning regarding potential declines or issues with visuo-cognitive function.

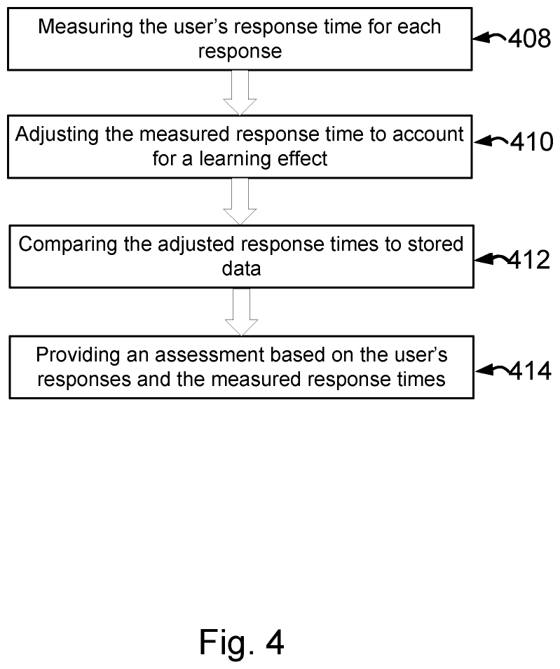

In some embodiments, the at least one attribute may include the response time (i.e. the time taken by the user to respond to the stimulus after it has been displayed on the screen), this is discussed in more detail in FIG. 4.

It will be appreciated that the at least one attribute does not include reading time, as the present disclosure is not directed to seeing how long the user takes to read a block text.

Similarly, the handheld computing device 100, 200 may comprise a motion sensor, such as an accelerometer. The motion sensor may detect movement of the handheld computing device 100, 200. Thus, in some embodiments the at least one attribute may include the movement of the handheld computing device at or around the time that the user's response is received. In some embodiments, the attribute may comprise a value indicative of any movement detected by the motion sensor in a predetermined time period before, after or either side of a response being received from the user. For example, if the handheld computing device is shaking or moving around then this may be an early indication of a decline in visual function, as people with weakening vision often wobble or move the screen to try to catch the light or change the location of the stimulus in their visual field, to try and improve their visual ability.

In some embodiments, the at least one attribute may include a distance between the user (or the user's eye) and the handheld computing device 100, 200. The distance may be measured by the camera 104. It will be appreciated that the user may move the display screen closer or further away from their eye (depending on their visual function) in order to try and improve their visual ability.

In some embodiments, the at least one attribute may include an order (or pattern) in which the user's responses are received. Each stimulus may be allocated a number or identifier indicative of the order in which a response from the user to that stimulus was detected.

For example, referring back to FIG. 1A, assuming that all of these stimuli 106 are presented on the screen at a given time, if the user has relatively good visual function they may respond to each stimulus 106 by starting at one side of the screen and moving across to the other side of the screen, responding to each of the stimuli 106 in the order that they are displayed on the screen (e.g. left to right: smiley face, large 6-point star, owl, 16-point star, sock, small 6-point star; or right to left: small 6-point star, sock, 16-point star, owl, large 6-point star, smiley face).

In comparison, if the user has relatively poor visual function, they may not respond to the stimuli in the same order as the user that has good visual function. Instead, the user may respond to the stimuli in order of how difficult they are for the user to see, starting with the easiest to see stimuli. Thus, the pattern or order in which the user responds to the stimuli can provide a useful insight into the visual function of the user. These stimuli may be presented specifically for the purpose of assessing visual function, but the text and iconography presented during the usual course of use of the mobile device may also be used for this purpose.

In some embodiments, the at least one attribute may include a measurement of the number or types of mistakes made by the user. As such, step 308 may include measuring the number of times that a user touches the display screen 102 at, or otherwise responds to, a location where a stimulus 106 is not presented. If a user frequently makes these mistakes, or makes a large number of these mistakes, it may indicate that they are randomly pressing or looking all around the screen, and not actually seeing the stimuli that are presented. This may indicate that the results of the assessment may not be very accurate or reliable, or that the user's visual function may be worse than is indicated (as some of the stimuli determined to be seen by the user may be false positives).

In some embodiments, the stimuli may be presented as part of a game, which may require the user to make a choice between different stimuli according to a given condition. For example, the user may be prompted, by the handheld computing device by audio and/or visual instructions, to select a stimulus that satisfies a given condition. Thus, the measurement of the number of types of mistakes made by the user may be a measurement of the number of incorrect stimuli selected by the user (i.e. the number of responses from the user that indicate a stimulus that does not satisfy the given condition). Whilst this type of game may not be suitable for all users, such as small children or people with severe learning difficulties, it may be useful for other users as it may gather useful information more quickly than other types of assessment.

In some embodiments, the at least one attribute may include a measurement of the accuracy of the responses received from the user. For example, this may include a ratio of the number of mistakes made by the user to the total number of responses received from the user during the assessment. Conversely, this may include a ratio of the number of stimuli determined to be seen by the user to the total number of responses received from the user during the assessment.

In step 310 the method includes recording the at least one attribute to enable an assessment of at least one aspect of the user's visuo-cognitive function. This may include storing the at attribute data in a memory of the handheld computing device, or transmitting this data to a remote server or computing device.

In some embodiments, a primary assessment may be provided of at least one aspect of the user's visuo-cognitive function based on whether or not the user responds to each stimulus. The determined at least one attribute of the user's responses may be used to provide a secondary assessment of at least one aspect of the user's visuo-cognitive function. In some embodiments, the secondary assessment may be a more general or qualitative (e.g. less specific) assessment of the user's visuo-cognitive function.

In some embodiments, the results from whether the user responds to each stimulus and the determined at least one attribute of the user's responses may be combined into a single assessment of the at least aspect of visuo-cognitive function. In some embodiments, the determined at least one attribute may be used to adjust or influence the assessment of the at least one aspect of visuo-cognitive function.

In an additional step (not shown in FIG. 3), the method may include comparing the measured data to stored data. The stored data may be stored in a memory of the handheld computing device, or retrieved from an external memory or storage resource.

In some embodiments, the method may include comparing the results of the assessment to stored data for the same user from previous assessments. Thus, the determined at least one attribute of the user's responses may be monitored over time. This may provide additional useful information compared to the stand-alone data.

For example, if the at least one attribute comprises the average distance of the handheld computing device from the user, by comparing this to past data associated with the same user, any change in habitual viewing distance may be detected. A change in habitual viewing distance may be an early indication of a change in visual function.

In another example, if the at least one attribute comprises a measurement of the accuracy of the user's responses, a change in the user's accuracy over time, such as a reduction in the consistency or accuracy of their responses, may be an early indication of a change in visual function.

In some embodiments, the stored data may comprise age-matched average data, to enable the user's results to be compared against the established average for a person of their age.

In step 312, the method (i.e. steps 302 to 310) is repeated for subsequent stimuli that are presented on the screen. In some embodiments, if a plurality of stimuli are presented on the screen at step 302, then step 312 may not be required. The method may optionally continue until a predetermined number of stimuli are presented to the user.

In step 314, the assessment (e.g. steps 302 to 312) may be repeated for the user's other eye. For example, the user may adjust the eye patch or other covering to cover their other eye and repeat the method detailed above. Thus, the user's visual function may be accessed for each eye separately.

FIG. 4 shows a flowchart of another method of assessing at least one aspect of visual function according to an embodiment of the present disclosure, which follows on from step 306 in FIG. 3.

In step 408 the method comprises measuring the user's response time for each response. As explained above, the response time is defined as the time elapsed from when the stimulus is first displayed to when the user's response to the stimulus is received. Thus, in this embodiment, the at least one attribute includes the response time. The method may optionally include the step of determining (or recording) one or more additional attributes of the user's responses (not shown in FIG. 4).

It will be appreciated that in some embodiments step 408 may actually occur before step 306, as the user's response time may be used (at least in part) to determine whether or not the user has seen (or responded to) the stimulus.

In step 410 the method comprises adjusting the measured response time (from step 408) to adjust for a learning effect. The first time that the user carries out the assessment using the handheld computing device they will be unfamiliar with the set-up and the requirements. Over time, the user will gain experience and familiarity with the assessment and how to respond to the stimuli etc. As such, at the first assessment the user's response time to a given stimulus will likely be longer than by the nth assessment, even if there is no change to the user's visual function. This is what is referred to as the ‘learning effect’ in the present disclosure. If this learning effect is not accounted for, the assessment may falsely report an improvement in the user's visuo-cognitive function over time.

In order to account for the learning effect, the system of the present disclosure may record the number of times that a given user has carried out the assessment of visual function, or the number of stimuli that have been presented to the user. This number may be associated with the user's profile. This number, or a value derived from this number, may then be used to scale the measured response times to account for the learning effect. Such an adjustment may be carried out by scaling the measured response time, for example using population level factors that reflect the average response time change with increasing numbers of assessments (or time since the first, or most recent, assessment) in the demographic to which the given user belongs.

At step 412 the method includes comparing the adjusted response times to stored data. As described above, the stored data may comprise data from previous assessments carried out by the user. Thus, this allows the user's response times to be monitored over time. If the user is taking longer on average to respond to the same stimuli, this may be an early indication of a decline in visuo-cognitive function, even if the user still responds to the stimulus. If, for example, the user is only taking longer to see certain stimuli, this can provide additional information regarding which aspects of the user's visual function are affected.

It will be appreciated that step 410 is an optional step, so if this step is not included the response times referred to in 412 will be the actual response time measured.

Optionally, the handheld computing device may output a warning or prompt to the user to seek specialist assistance, for example if the user's response time has deviated from their historical average response time by more than a predetermined threshold.

At step 414 the method comprises providing an assessment based on the user's responses and the measured response times.

It will be appreciated that following step 414 the assessment may be repeated for the user's other eye.

Accordingly, there has been described computer-implemented systems and methods for assessing at least one aspect of a user's visuo-cognitive function, comprising presenting a plurality of stimuli on a display screen of a handheld computing device, detecting a response, or a lack of response, from the user to each stimulus, determining at least one attribute associated with the user's responses, wherein each attribute is indicative of a level of difficulty associated with seeing or processing the plurality of stimuli, and recording the at least one attribute to enable an assessment of at least one aspect of a user's visuo-cognitive function.

Although particular embodiments of this disclosure have been described, it will be appreciated that many modifications/additions and/or substitutions may be made within the scope of the claims.

### Illustrative Computing Environment

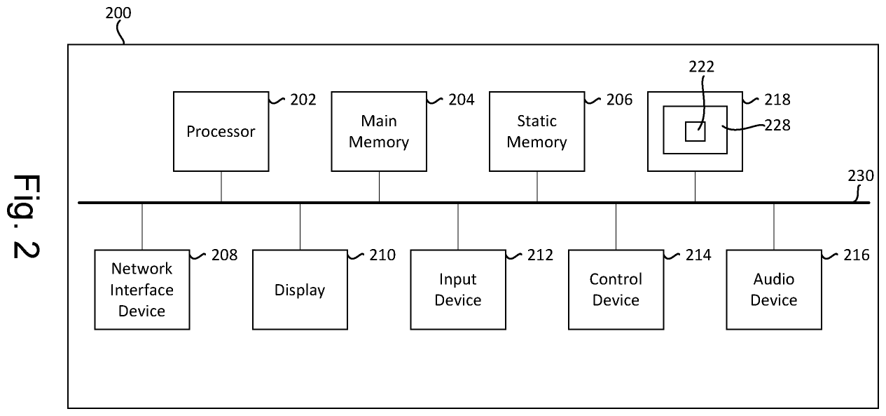

FIG. 2 illustrates a possible computing environment for putting of an illustrative embodiment of the disclosure into effect. In particular, FIG. 2 provides a block diagram of one example implementation of a handheld computing device 200 according to the present disclosure. The handheld computing device 200 may be the same as the handheld computing device 100 in FIG. 1A or 1B. The handheld computing device 200 is associated with executable instructions for causing the handheld computing device 200 to perform any one or more of the methodologies discussed herein. The handheld computing device 200 may operate in the capacity of the data model or one or more computing resources for implementing the data model for carrying out the methods of the present disclosure. In alternative implementations, the handheld computing device 200 may be connected (e.g., networked) to other machines in a Local Area Network (LAN), an intranet, an extranet, or the Internet. The computing device may operate in the capacity of a server or a client machine in a client-server network environment, or as a peer machine in a peer-to-peer (or distributed) network environment.

The example handheld computing device 200 includes a processing device 202, a main memory 204 (e.g., read-only memory (ROM), flash memory, dynamic random-access memory (DRAM) such as synchronous DRAM (SDRAM) or Rambus DRAM (RDRAM), etc.), a static memory 206 (e.g., flash memory, static random-access memory (SRAM), etc.), and a secondary memory (e.g., a data storage device 218), which communicate with each other via a bus 230.

Processing device 202 represents one or more general-purpose processors such as a microprocessor, central processing unit, or the like. More particularly, the processing device 202 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 202 may also be one or more special-purpose processing devices such as an application specific integrated circuit (ASIC), a field programmable gate array (FPGA), a digital signal processor (DSP), network processor, or the like. Processing device 202 is configured to execute the processing logic (instructions 222) for performing the operations and steps discussed herein.

The handheld computing device 200 may further include a network interface device 208. The handheld computing device 200 also includes a display unit 210 (e.g., a liquid crystal display (LCD) or a cathode ray tube (CRT)), an alphanumeric input device 212 (e.g., a keyboard or touchscreen), a cursor control device 214 (e.g., touchscreen), and optionally an audio device 216 (e.g., a speaker).

The data storage device 218 may include one or more machine-readable storage media (or more specifically one or more non-transitory computer-readable storage media) 228 on which is stored one or more sets of instructions 222 embodying any one or more of the methodologies or functions described herein. The instructions 222 may also reside, completely or at least partially, within the main memory 204 and/or within the processing device 202 during execution thereof by the computer system 200, the main memory 204 and the processing device 202 also constituting computer-readable storage media.

The various methods described above may be implemented by a computer program. The computer program may include computer code arranged to instruct a computer to perform the functions of one or more of the various methods described above. The computer program and/or the code for performing such methods may be provided to an apparatus, such as a computer, on one or more computer readable media or, more generally, a computer program product. The computer readable media may be transitory or non-transitory. The one or more computer readable media could be, for example, an electronic, magnetic, optical, electromagnetic, infrared, or semiconductor system, or a propagation medium for data transmission, for example for downloading the code over the Internet. Alternatively, the one or more computer readable media could take the form of one or more physical computer readable media such as semiconductor or solid-state memory, magnetic tape, a removable computer diskette, a random-access memory (RAM), a read-only memory (ROM), a rigid magnetic disc, and an optical disk, such as a CD-ROM, CD-R/W or DVD.

In an implementation, the modules, components and other features described herein can be implemented as discrete components or integrated in the functionality of hardware components such as ASICS, FPGAs, DSPs or similar devices.

A “hardware component” is a tangible (e.g., non-transitory) physical component (e.g., a set of one or more processors) capable of performing certain operations and may be configured or arranged in a certain physical manner. A hardware component may include dedicated circuitry or logic that is permanently configured to perform certain operations. A hardware component may be or include a special-purpose processor, such as a field programmable gate array (FPGA) or an ASIC. A hardware component may also include programmable logic or circuitry that is temporarily configured by software to perform certain operations.

Accordingly, the phrase “hardware component” should be understood to encompass a tangible entity that may be physically constructed, permanently configured (e.g., hardwired), or temporarily configured (e.g., programmed) to operate in a certain manner or to perform certain operations described herein.

In addition, the modules and components can be implemented as firmware or functional circuitry within hardware devices. Further, the modules and components can be implemented in any combination of hardware devices and software components, or only in software (e.g., code stored or otherwise embodied in a machine-readable medium or in a transmission medium).

Unless specifically stated otherwise, as apparent from the following discussion, it is appreciated that throughout the description, discussions utilizing terms such as “providing”, “calculating”, “computing,” “identifying”, “detecting”, “establishing”, “training”, “determining”, “storing”, “generating” ,“checking”, “obtaining” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

### Enumerated Clauses

We now provide enumerated clauses which illustrate, without limitation, example embodiments of the present disclosure. Any feature provided below in respect of one clause may be incorporated into or combined with any other clause(s).
Clause 1. A computer-implemented method for assessing at least one aspect of a user's visuo-cognitive function, the method comprising:
   presenting a plurality of stimuli on a display screen of a (handheld) computing device or system;
   detecting at least one response, or lack of response, from the user to the plurality of stimuli;
   determining at least one attribute associated with the user's response(s), wherein each attribute is indicative of or related to a level of difficulty associated with seeing or processing the plurality of stimuli; and
   recording the at least one attribute to enable an assessment of at least one aspect of a user's visuo-cognitive function.
Clause 2. The computer-implemented method of clause 1, further comprising:
   i) altering the plurality of stimuli to change the level of difficulty associated with seeing or processing the plurality of stimuli;
   ii) presenting the altered plurality of stimuli on the display screen of the (handheld) computing device or system, and
   iii) determining at least one attribute associated with the user's response(s) to the altered plurality of stimuli.

Preferably, altering the plurality of stimuli comprises reducing the level of difficulty such that the plurality of stimuli becomes easier to see or process by the user.
Clause 3. The computer-implemented method of clause 1 or 2, further comprising:
   providing an assessment of at least one aspect of the user's visuo-cognitive function at least partially based on the at least one attribute.
Clause 4. The computer-implemented method of clauses 1, 2 or 3, wherein the at least one attribute comprises one or more of:
   a response time or average response time;
   a number of incorrect responses received from the user;
   an indication of a pattern or order of the responses received;
   an amount of movement of the (handheld) computing device or system relative to a user's eye when the response is received; and/or
   a distance between the (handheld) computing device, system or the display screen and the user or the user's eye when the response is received or not received.
Clause 5. The computer-implemented method of any preceding clause, wherein detecting a response from the user comprises receiving an input from the user indicating a proposed location of the respective stimulus on the display screen.
Clause 6. The computer-implemented method of clause 5, wherein the at least one attribute comprises:
   an accuracy of the proposed location compared to an actual location of the stimulus on the display screen.
Clause 7. The computer-implemented method of clause 5 or clause 6, further comprising: determining whether the response from the user indicates that the user has seen the respective stimulus by determining whether the proposed location of the stimulus is within a predetermined range of an actual location of the stimulus on the display screen.
Clause 8. The computer-implemented method of any preceding clause, wherein determining 5 the at least one attribute comprises: determining a response time for each response, wherein the response time is the time elapsed between a display time when the stimulus is presented on the display screen and a time that the response from the user is detected.
Clause 9. The computer-implemented method of any preceding clause, further comprising: adjusting the determined response time to account for a learning effect.
Clause 10. The computer-implemented method of clause 9, further comprising:
   recording a value indicative of a number of assessments that have been completed by the user, or a number of stimuli that have been presented to the user; and
   applying a scaling formula to the determined response time to account for the learning effect, wherein the scaling formula is based at least in part on the value.
Clause 11. The computer-implemented method of any preceding clause, further comprising:
   comparing the determined at least one attribute against stored data; and/or monitoring the determined at least one attribute over a period of time, or over a number of assessments carried out by the user.
Clause 12. The computer-implemented method of clause 11, wherein comparing the determined at least one attribute against stored data comprises:
   comparing the determined at least one attribute against stored data for said user; and/or comparing the determined at least one attribute against an age normative database comprising age-matched average data.
Clause 13. The computer-implemented method of any preceding clause, wherein determining the at least one attribute comprises:
   determining any incorrect responses received from the user, wherein each incorrect response indicates that the user has not seen the stimulus, or has not seen one of the plurality of stimuli.
Clause 14. The computer-implemented method of clause 13, further comprising:
   recording at least one of a time of receipt and a location on the display screen of each incorrect response from the user; and/or determining an accuracy of the responses received from the user, based on the number of incorrect responses received.
Clause 15. The computer-implemented method of any preceding clause, wherein determining the at least one attribute comprises:
   measuring movement of the (handheld) computing device or system when each response from the user is detected; and/or determining a distance between the user, or the user's eye, and the display screen when each response from the user is detected.
Clause 16. The computer-implemented method of any preceding clause, wherein determining the at least one attribute comprises:
   determining an order or pattern in which the user's responses to the plurality of stimuli are received; and/or
   determining a location on the display screen or device at which the user was targeting their gaze when each response from the user is detected; and/or
   determining the user's facial expression during the time in which the user is identifying and/or responding to the stimulus.
Clause 17. The computer-implemented method of any preceding clause, wherein the plurality of stimuli comprises:
   stimuli which are presented specifically for the purpose of assessment of visuo-cognitive function; and/or display elements typically encountered during the normal usage of the handheld computing device, or modifications thereof.
Clause 18. The computer-implemented method of any preceding clause, wherein each stimulus has at least one respective visual property and wherein:
   the at least one visual property has a predetermined value selected to assess at least one aspect of visuo-cognitive function; and/or the at least one visual property comprises one or more of: position on the display screen; contrast; colour; level of detail; shape; size; or movement of the stimulus on the display screen.
Clause 19. The computer-implemented method of any preceding clause,
   wherein one or more of the plurality of stimuli are repeatedly presented on the display screen and a response, or lack of response, from the user is detected at each presentation; and/or
   wherein the plurality of stimuli are presented, or repeatedly presented, on the display screen until a threshold is determined for the at least one aspect of the user's visuo-cognitive function, further comprising comparing the at least one attribute associated with the user's response to a stimulus near the user's threshold for a given aspect of visuo-cognitive function, to the at least one attribute associated with the user's response to a stimulus above the user's threshold for the same aspect of visuo-cognitive function.
Clause 20. The computer-implemented method of any preceding clause, wherein the display screen comprises a touchscreen, and wherein detecting a response from the user comprises detecting a point of contact from the user on the touchscreen.
Clause 21. The computer-implemented method of any preceding clause, wherein detecting a response from the user comprises monitoring movement of the user's eye using a camera.
Clause 22. The computer-implemented method of any preceding clause, comprising presenting the plurality of stimuli on the display screen within a gamified environment.
Clause 23. The computer-implemented method of any preceding clause, further comprising:
   storing the determined at least one in a memory; and/or
   transmitting the determined at least one attribute to a remote server and/or a remote electronic device via a wireless communication channel.
Clause 24. The computer-implemented method of any preceding clause, comprising executing the assessment for a first eye of the user with the user's second eye being covered, and then repeating the assessment for the user's second eye with the first eye being covered.
Clause 25. A computing device or system for assessing at least one aspect of a user's visuo-cognitive function, the handheld computing device comprising:
   a display screen;
   a processor; and
      memory including executable instructions that, as a result of execution by the processor, causes the handheld computing device to perform the computer-implemented method of any of clauses 1 to 24.

The computing device or system may comprise a handheld component/computing device.
Clause 26. The computing device or system of clause 25, wherein the display screen comprises a touchscreen, wherein to detect a response from the user the computing device/system is configured to detect a point of contact from the user on the touchscreen.
Clause 27. The handheld computing device of clause 25 or clause 26, wherein the computing device/system comprises a camera, wherein the camera is configured to monitor movement of the user's eye to detect a response from the user.

1. A computer-implemented method for assessing at least one aspect of a user's visuo-cognitive function, the method comprising:
presenting a plurality of stimuli on a display screen of a handheld computing device;
detecting at least one response, or lack of response, from the user to the plurality of stimuli;
determining at least one attribute associated with the user's response(s), wherein each attribute is indicative of or related to a level of difficulty associated with seeing or processing the plurality of stimuli; and
recording the at least one attribute to enable an assessment of at least one aspect of a user's visuo-cognitive function.

2. The computer-implemented method of claim 1, further comprising:
i) altering the plurality of stimuli to change the level of difficulty associated with seeing or processing the plurality of stimuli;
ii) presenting the altered plurality of stimuli on the display screen of the handheld computing device, and
iii) determining at least one attribute associated with the user's response(s) to the altered plurality of stimuli.

3. The computer-implemented method of claim 1, further comprising:
providing an assessment of at least one aspect of the user's visuo-cognitive function at least partially based on the at least one attribute.

4. The computer-implemented method of claim 1, wherein the at least one attribute comprises one or more of:
a response time or average response time;
a number of incorrect responses received from the user;
an indication of a pattern or order of the responses received;
an amount of movement of the handheld computing device relative to a user's eye when the response is received; and/or
a distance between the handheld computing device and the user or the user's eye when the response is received.

5. The computer-implemented method of claim 1, wherein detecting a response from the user comprises receiving an input from the user indicating a proposed location of the respective stimulus on the display screen.

6. The computer-implemented method of claim 5, wherein:
i) the at least one attribute comprises: an accuracy of the proposed location compared to an actual location of the stimulus on the display screen; or
ii) the method further comprises determining whether the response from the user indicates that the user has seen the respective stimulus by determining whether the proposed location of the stimulus is within a predetermined range of an actual location of the stimulus on the display screen.

7. (canceled)

8. The computer-implemented method of claim 1, wherein determining the at least one attribute comprises: determining a response time for each response, wherein the response time is the time elapsed between a display time when the stimulus is presented on the display screen and a time that the response from the user is detected.

9. The computer-implemented method of claim 1, further comprising: adjusting the determined response time to account for a learning effect.

10. The computer-implemented method of claim 9, further comprising:
recording a value indicative of a number of assessments that have been completed by the user, or a number of stimuli that have been presented to the user; and
applying a scaling formula to the determined response time to account for the learning effect, wherein the scaling formula is based at least in part on the value.

11. The computer-implemented method of claim 1, further comprising:
comparing the determined at least one attribute against stored data; and/or
monitoring the determined at least one attribute over a period of time, or over a number of assessments carried out by the user.

12. The computer-implemented method of claim 11, wherein comparing the determined at least one attribute against stored data comprises:
comparing the determined at least one attribute against stored data for said user; and/or
comparing the determined at least one attribute against an age normative database comprising age-matched average data.

13. The computer-implemented method of claim 1, wherein determining the at least one attribute comprises: determining any incorrect responses received from the user, wherein each incorrect response indicates that the user has not seen the stimulus, or has not seen one of the plurality of stimuli.

14. The computer-implemented method of claim 13, further comprising:
recording at least one of a time of receipt and a location on the display screen of each incorrect response from the user; and/or
determining an accuracy of the responses received from the user, based on the number of incorrect responses received.

15. The computer-implemented method of claim 1, wherein determining the at least one attribute comprises:
i) measuring movement of the handheld computing device when each response from the user is detected; and/or
determining a distance between the user, or the user's eye, and the display screen when each response from the user is detected; or
ii) determining an order or pattern in which the user's responses to the plurality of stimuli are received; and/or
determining a location on the display screen or device at which the user was targeting their gaze when each response from the user is detected; and/or
determining the user's facial expression during the time in which the user is identifying and/or responding to the stimulus.

16. (canceled)

17. The computer-implemented method of claim 1,
wherein:
i) the plurality of stimuli comprises:
stimuli which are presented specifically for the purpose of assessment of visuo-cognitive function; and/or
display elements typically encountered during the normal usage of the handheld computing device, or modifications thereof;
or
ii) each stimulus has at least one respective visual property and wherein:
the at least one visual property has a predetermined value selected to assess at least one aspect of visuo-cognitive function; and/or
the at least one visual property comprises one or more of: position on the display screen;
contrast; colour; level of detail; shape; size; or movement of the stimulus on the display screen.

18. (canceled)

19. The computer-implemented method of claim 1,
wherein one or more of the plurality of stimuli are repeatedly presented on the display screen and a response, or lack of response, from the user is detected at each presentation; and/or
wherein the plurality of stimuli are presented, or repeatedly presented, on the display screen until a threshold is determined for the at least one aspect of the user's visuo-cognitive function, further comprising comparing the at least one attribute associated with the user's response to a stimulus near the user's threshold for a given aspect of visuo-cognitive function, to the at least one attribute associated with the user's response to a stimulus above the user's threshold for the same aspect of visuo-cognitive function.

20. The computer-implemented method of claim 1,
wherein one or more applies of:
i) the display screen comprises a touchscreen, and wherein detecting a response from the user comprises detecting a point of contact from the user on the touchscreen;
ii) detecting a response from the user comprises monitoring movement of the user's eye using a camera; or
iii) presenting the plurality of stimuli on the display screen within a gamified environment.

21. (canceled)

22. (canceled)

23. The computer-implemented method of claim 1, further comprising:
i) storing the determined at least one in a memory; and/or
transmitting the determined at least one attribute to a remote server and/or a remote electronic device via a wireless communication channel; or
ii) executing the assessment for a first eye of the user with the user's second eye being covered, and then repeating the assessment for the user's second eye with the first eye being covered.

24. (canceled)

25. A handheld computing device for assessing at least one aspect of a user's visuo-cognitive function, the handheld computing device comprising:
a display screen;
a processor; and
memory including executable instructions that, as a result of execution by the processor, causes the handheld computing device to perform the computer-implemented method of claim 1.

26. The handheld computing device of claim 25, wherein
i) the display screen comprises a touchscreen, wherein to detect a response from the user the handheld computing device is configured to detect a point of contact from the user on the touchscreen; or
ii) the handheld computing device comprises a camera, wherein the camera is configured to monitor movement of the user's eye to detect a response from the user.

27. (canceled)
